(19) United States
(12) Patent Application Publication
(10) Pub. No.: US 2021/0369751 A1
(43) Pub. Date: Dec. 2, 2021

(54) BIOLOGICALLY ACTIVE COMPOSITIONS AND METHODS OF USING

(71) Applicant: Mediwel Labs, LLC, Bloomfield, IL (US)

(72) Inventor: Brett Park, Bloomfield, NJ (US)

(21) Appl. No.: 17/337,378

(22) Filed: Jun. 2, 2021

Related U.S. Application Data
(60) Provisional application No. 63/033,722, filed on Jun. 2, 2020.

(51) Int. Cl. A61K 31/706; A61K 31/4525; A61K 31/352; A61K 31/7048; A61K 31/7016; A61K 31/455; A61K 45/06

(52) U.S. Cl. CPC A61K 31/706; A61K 31/4525; A61K 31/352; A61K 45/06; A61K 31/7016; A61K 31/455; A61K 31/7048

(57) ABSTRACT

Disclosed herein is a composition comprising a biologically active amount of nicotinamide mononucleotide or a derivative thereof, apigenin or a derivative thereof, quercetin or a derivative thereof, and bioperine or a derivative thereof. A method of treating a physiological condition using the composition is also disclosed.

### RELATED APPLICATIONS

This application claims the benefit of a U.S. Provisional Application Ser. No. 63/033,722 filed Jun. 2, 2020, the disclosure of which is incorporated by reference herein in its entirety.

### BACKGROUND

The statements in this section merely provide background information related to the present disclosure and may not constitute prior art.

The field of anti-aging is replete with various supplements and nutraceuticals which claim to offer a way forward in mitigating the effects of aging on the human body.

It is now acknowledged that aging itself is deemed to be the leading risk factor to various diseases and co-morbidities. Many scientists now agree that this risk factor plays out over the course of the aging timeline. A timeline that we now associate with the biological clock or our genetic clock that seems to unwind and manifest by activating and deactivating our genes. There is now even a way to measure this genetic timeline and possible predictor of age-related onsets whether they be the potential risks for disease states or ultimately the timeline associated with our own lifespan. In that capacity, it is the well maintenance and health of our DNA and its ability to maintain and heal itself that is now the focus of much of our anti-aging endeavors.

Numerous flavonoids and other naturally occurring compounds are known in the art. However, the effectiveness of such materials to address the effects of aging have been largely ignored due to lower efficacy than more traditional synthetic organic compounds. There is a need in the art for improved medicines, especially those from naturally occurring sources.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

The instant disclosure is directed to a biologically active composition comprising a nicotinamide adenine dinucleotide (NAD) precursor nicotinamide mononucleotide or a derivative thereof, (NMN), in combination with inhibitors of CD38, apigenin or a derivative thereof and quercetin or a derivative thereof. The composition further includes an absorption enhancer piperine (also known as bioperine).

In other embodiments, a composition comprises a biologically active amount of nicotinamide mononucleotide or a derivative thereof, apigenin or a derivative thereof, quercetin or a derivative thereof, and bioperine or a derivative thereof.

In still other embodiments, a composition comprises a biologically active amount of trans-resveratrol or a derivative thereof, apigenin or a derivative thereof, quercetin or a derivative thereof, and bioperine or a derivative thereof.

In embodiments, a method to treat a physiological condition of a mammal comprises the steps of providing a composition according to one or more embodiments disclosed herein, and administering a biologically effective dosage of the composition to the mammal.

### DETAILED DESCRIPTION

At the outset, it should be noted that in the development of any such actual embodiment, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure. In addition, the composition used/disclosed herein can also comprise some components other than those cited. In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific, it is to be understood that inventors appreciate and understand that any and all data points within the range are to be considered to have been specified, and that inventors possessed knowledge of the entire range and all points within the range.

The following definitions are provided in order to aid those skilled in the art in understanding the detailed description.

As used in the specification and claims, “near” is inclusive of “at.” A hydrocarbon refers to a compound comprising carbon and hydrogen, whether saturated or unsaturated. A halogenated hydrocarbon refers to a hydrocarbon wherein at least one hydrogen is substituted with a halogen, F, Cl, Br, and/or I. An aqueous liquid refers to a liquid comprising from about 5 wt % up to 100 wt % water.

For the purposes of this invention and the claims thereto, the new numbering scheme for the Periodic Table Groups is used as described in Chemical and Engineering News, 63(5), pg. 27 (1985). Therefore, a “group 4 metal” is an element from group 4 of the Periodic Table, e.g. Hf, Ti, or Zr.

As used herein, and unless otherwise specified, the term “Cₙ” means hydrocarbon(s) having n carbon atom(s) per molecule, where n is a positive integer. Likewise, a “Cₘ-C_{y}” group or compound refers to a group or compound comprising carbon atoms at a total number thereof in the range from m to y. Thus, a C₁-C₄ alkyl group refers to an alkyl group that includes carbon atoms at a total number thereof in the range of 1 to 4, e.g., 1, 2, 3 and 4.

An electron neutral molecule refers to a molecule having a formal charge of zero (0). Accordingly, in an electron neutral molecule, the number of valence electrons is equal to the number of valence electrons possible around the atoms in the molecule. Likewise, the number of substituents required to make a molecule represented by a structure electron neutral is the total number of possible for the particular arrangement. For example, in the following structure:

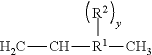

to make the molecule electron neutral when the R¹ moiety is a single oxygen atom, y=0, when the R¹ moiety is a single nitrogen atom, y=1, when the R¹ moiety is a single carbon atom, y=2; when the R¹ moiety has 2 carbon atoms, y=4; and so on.

The terms “group,” “radical,” and “substituent” may be used interchangeably.

The terms “hydrocarbyl radical,” “hydrocarbyl group,” or “hydrocarbyl” may be used interchangeably and are defined to mean a group consisting of hydrogen and carbon atoms only. Preferred hydrocarbyls are C₁-C₁₀₀ radicals that may be linear, branched, or cyclic, and when cyclic, aromatic or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl naphthyl, and the like.

Unless otherwise indicated, a functional group refers to a mono-valent, divalent, and/or trivalent moiety comprising elements from Group 13-17 of the periodic table of the elements and includes a hydrocarbyl, as substituted hydrocarbyl, a heteroatom, or a heteroatom containing group, such as halogen (such as Br, Cl, F or I) or at least one functional group such as —NR*₂, —NR*—CO—R*, —OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)_{q}—SiR*₃, or a combination thereof, where q is 1 to 10 and each R* is independently hydrogen, a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

The term “substituted hydrocarbyl” means a hydrocarbyl radical in which at least one hydrogen atom of the hydrocarbyl radical has been substituted with at least one heteroatom (such as halogen, e.g., Br, Cl, F or I) or heteroatom-containing group (such as a functional group, such as —NR*₂, —NR*—CO—R*, —OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)_{q}—SiR*₃, or a combination thereof, where q is 1 to 10 and each R* is independently hydrogen, a hydrocarbyl or halocarbyl radical, and two or more R* may join together to form a substituted or unsubstituted completely saturated, partially unsaturated, or aromatic cyclic or polycyclic ring structure), or where at least one heteroatom has been inserted within a hydrocarbyl ring.

A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom substituted ring. A substituted heterocyclic ring is a heterocyclic ring where a hydrogen of one of the ring atoms is substituted, e.g., replaced with a hydrocarbyl, or a heteroatom containing group (as further described in the definition of “substituted” herein).

The following abbreviations may be used herein: Me is methyl, Ph is phenyl.

A glycoside derivative of a compound refers to a molecule in which a sugar is bound to the particular compound, typically via another functional group, via a glycosidic bond. Glycosides can be linked by an O— (an O-glycoside), N-(a glycosylamine), S-(a thioglycoside), or C-(a C-glycoside) glycosidic bond. In the IUPAC naming scheme, the name “C-glycoside” is referred to as a “C-glycosyl compound”. For purposes herein, in a first instance all stereochemical configurations are assumed present individually, and in a second instance any combination of stereochemical configurations are assumed present for purposes of this disclosure. For example, the following is a glycoside of apigenin.

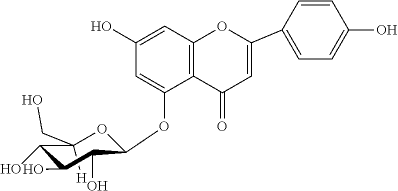

The term “alkenyl” means a straight-chain, branched-chain, or cyclic hydrocarbon radical having one or more double bonds. These alkenyl radicals may be optionally substituted. Examples of suitable alkenyl radicals include ethenyl, propenyl, allyl, 1,4-butadienyl cyclopropenyl, cyclobutenyl, cyclopentenyl, cyclohexenyl, cyclooctenyl, including their substituted analogues.

The term “alkoxy” or “alkoxide” means an alkyl ether radical wherein the term alkyl is as defined above. Examples of suitable alkyl ether radicals include methoxy, ethoxy, n-propoxy, iso-propoxy, n-butoxy, iso-butoxy, sec-butoxy, tert-butoxy, and phenoxyl.

The term “aryl” or “aryl group” includes a C₄-C₂₀ aromatic ring, such as a six carbon aromatic ring, and the substituted variants thereof, including phenyl, 2-methyl-phenyl, xylyl, 4-bromo-xylyl. Likewise, heteroaryl means an aryl group where a ring carbon atom (or two or three ring carbon atoms) has been replaced with a heteroatom, such as N, O, or S. As used herein, the term “aromatic” also refers to pseudoaromatic heterocycles which are heterocyclic substituents that have similar properties and structures (nearly planar) to aromatic heterocyclic ligands, but are not by definition aromatic; likewise, the term aromatic also refers to substituted aromatics.

The terms “aryloxy” and “aryloxide” mean an aryl group bound to an oxygen atom, such as an aryl ether group/radical connected to an oxygen atom and can include those where the aryl group is a C₁ to C₁₀ hydrocarbyl. Examples of suitable aryloxy radicals can include phenoxy, and the like.

The terms “hydrosilylcarbyl radical,” “hydrosilylcarbyl group,” or “hydrosilylcarbyl” interchangeably refers to a group consisting of hydrogen, carbon, and silicon atoms only. A hydrosilylcarbyl group can be saturated or unsaturated, linear or branched, cyclic or acyclic, aromatic or non-aromatic, and with the silicon atom being within and/or pendant to the cyclic/aromatic rings.

The term “silyl group,” refers to a group comprising silicon atoms, such as a hydrosilylcarbyl group.

For purposes herein, a “ring carbon atom” is a carbon atom that is part of a cyclic ring structure. By this definition, a benzyl group has six ring carbon atoms and para-methylstyrene also has six ring carbon atoms.

The term “ring atom” means an atom that is part of a cyclic ring structure. By this definition, a benzyl group has six ring atoms, all of which are carbon, and tetrahydrofuran has 5 ring atoms, 4 carbon ring atoms and one oxygen ring atom.

Where isomers of a named alkyl, alkenyl, alkoxide, or aryl group exist (e.g., n-butyl, iso-butyl, iso-butyl, and tert-butyl) reference to one member of the group (e.g., n-butyl) shall expressly disclose the remaining isomers (e.g., iso-butyl, sec-butyl, and tert-butyl) in the family. Likewise, reference to an alkyl, alkenyl, alkoxide, or aryl group without specifying a particular isomer (e.g., butyl) expressly discloses all isomers (e.g., n-butyl, iso-butyl, sec-butyl, and tert-butyl).

For any particular compound disclosed herein, any general or specific structure presented also encompasses all conformational isomers, regio-isomers, and stereoisomers that may arise from a particular set of substituents, unless stated otherwise. Similarly, unless stated otherwise, the general or specific structure also encompasses all enantiomers, diastereomers, and other optical isomers whether in enantiomeric or racemic forms, as well as mixtures of stereoisomers, as would be recognized by a skilled artisan.

For purposes herein, a stilbenoid refers to a hydroxylated derivative of stilbene having a C₆-C₂-C₆ structure, belonging to the family of phenylpropanoids.

Alkaloids refer to naturally occurring organic compounds that comprise one or more basic nitrogen atoms. This group may also include some related compounds with neutral and even weakly acidic properties according to common understanding in the art. In addition to carbon, hydrogen and nitrogen, alkaloids may also contain oxygen, sulfur and, more rarely, other elements such as chlorine, bromine, and phosphorus.

As used herein, flavonoids refer to polyphenolic molecules having a 15 carbon skeleton, which are typically soluble in water. In embodiments, the flavonoids comprise two benzene rings connected by a carbon chain, typically a three carbon chain. One of the carbons in this chain is connected to a carbon in one of the benzine rings, either through an ether likage (an oxygen bridge) or directly to the phenyl ring, forming a third middle ring according to the general formula:

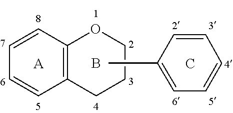

wherein the aliphatic bridge between rings B and C is typically bonded to the #2, #3, and/or #4 position on ring B. Likewise, an oxygen containing functional group is typically bound to one or more of the #2, #3, and/or #4 position on ring B.

The flavonoids can be divided into six major subtypes, which include chalcones, flavones, isoflavonoids, flavanones, anthoxanthins and anthocyanins.

Trans-resveratrol refers to (3,5,4′-trihydroxy-trans-stilbene), naturally occurring phenol according to the general formula:

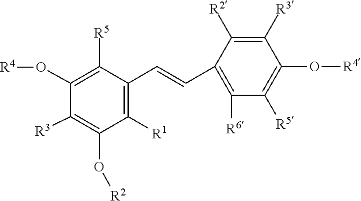

wherein each of R¹, R², R³, R⁴, R⁵, R^{2′}, R^{3′}, R^{4′}, R^{5′}, and R^{6′} are hydrogen. In embodiments, the trans-resveratrol is present as a derivative, which for purposes herein is defined as having the above structure, wherein one or more of R¹, R², R³, R⁴, R⁵, R^{2′}, R^{3′}, R^{4′}, R^{5′}, and R^{6′} are substituted with a C₁-C₄₀ hydrocarbyl radical, a substituted hydrocarbyl radical, a functional group comprising elements from Group 13-17 of the periodic table of the elements, or wherein two or more of R¹, R², R³, R⁴, R⁵, R^{2′}, R^{3′}, R^{4′}, R^{5′}, and R^{6′} independently join together to form a C₄ to C₆₂ cyclic or polycyclic ring structure.

Apigenin refers to a compound having the general structure

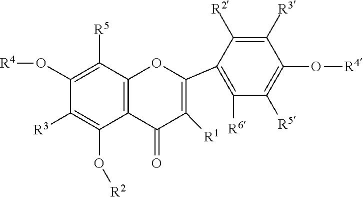

wherein each of R¹, R², R³, R⁴, R⁵, R^{2′}, R^{3′}, R^{4′}, R^{5′}, and R^{6′} are hydrogen. In embodiments, the apigenin is present as a derivative, which for purposes herein is defined as having the above structure, wherein one or more of R¹, R², R³, R⁴, R⁵, R^{2′}, R^{3′}, R^{4′}, R^{5′}, and R^{6′} are substituted with a C₁-C₄₀ hydrocarbyl radical, a substituted hydrocarbyl radical, a functional group comprising elements from Group 13-17 of the periodic table of the elements, or wherein two or more of R¹, R², R³, R⁴, R⁵, R^{2′}, R^{3′}, R^{4′}, R^{5′}, and R^{6′} independently join together to form a C₄ to C₆₂ cyclic or polycyclic ring structure.

Quercetin refers to a compound having the general structure:

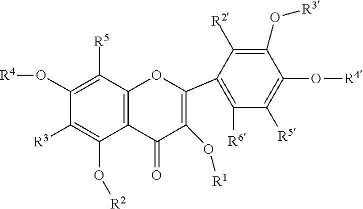

wherein each of R¹, R², R³, R⁴, R⁵, R^{2′}, R^{3′}, R^{4′}, R^{5′}, and R^{6′} are hydrogen. In embodiments, the quercetin is present as a derivative, which for purposes herein is defined as having the above structure, wherein one or more of R¹, R², R³, R⁴, R⁵, R^{2′}, R^{3′}, R^{4′}, R^{5′}, and R^{6′} are substituted with a C₁-C₄₀ hydrocarbyl radical, a substituted hydrocarbyl radical, a functional group comprising elements from Group 13-17 of the periodic table of the elements, or wherein two or more of R¹, R², R³, R⁴, R⁵, R^{2′}, R^{3′}, R^{4′}, R^{5′}, and R^{6′} independently join together to form a C₄ to C₆₂ cyclic or polycyclic ring structure.

Bioperine, also referred to in the art as piperine, is an alkaloid having the general structure:

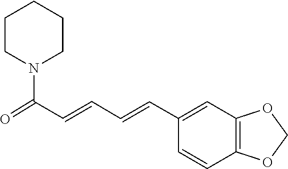 [SMILES: O=C(/C=C/C=C/c1ccc2c(c1)OCO2)N1CCCCC1]

In some embodiments, any liable carbon, nitrogen, and/or oxygen atom may be further substituted as described above to form a derivative thereof. For purposes herein, bioperine and piperine are used interchangeably throughout.

### Biologically Active Composition

The field of anti-aging is replete with various supplements and nutraceuticals which claim to offer a way forward in mitigating the effects of aging on the human body. It is now acknowledged that aging itself is deemed to be the leading risk factor to various diseases and co-morbidities. Those of skill in the art agree that this risk factor plays out over the course of the aging timeline. A timeline that we now associate with the biological clock or our genetic clock that seems to unwind and manifest by activating and deactivating our genes. There is now even a way to measure this genetic timeline and possible predictor of age-related onsets whether they be the potential risks for disease states or ultimately the timeline associated with one's own lifespan. In that capacity, it is the well maintenance and health of our DNA and its ability to maintain and heal itself that is now the focus of much of our anti-aging endeavors.

It is in addressing these issues where the study of the human genome and aging has converged to foster a new field of antiaging tied to active ingredients and activities for healthier aging. Compositions according to embodiments disclosed herein are directed to improvements based on research tied to NAD and its critical role as the co-enzyme to nearly all aspects of our cellular existence. It is this nicotinamide adenine dinucleotide (NAD) and the means to enhance its availability in the body to which the instant compositions are directed. It lends itself to a complex set of mechanism to raise the body's availability of NAD+(the activated form of NAD), instructive of the crucial metabolic process which occurs every cell to maintain life itself. It is especially the case as it relates to the health, stability, and the regeneration of our cellular DNA, including the process tied to a group of proteins called poly (ADP-ribose) polymerase known as PARP which functions to repair and maintain, while the activation of genetic signaling proteins known as Sirtuins is now at the center of healthy homeostasis as it relates to DNA stability.

Embodiments of the instant composition are directed to benefits obtained from the greater understanding of the highly complex sequences of locking and unlocking the genetic codes that are associated with protein sequencing for all things cellular, which is by nature, a highly energy consuming process. It is also one where there are a number of different supporting protein mechanisms by which genes are signaled to unwind and release, and which to keep in place for future use. For this to happen without failure, they all require the critical availability of NAD+. The lack of NAD+, and its deficit is tied to the deleterious effects of old age and aging itself, as there are over 524 enzymatic activities required its metabolic use, just for the body to survive. But as we age there is steep decline in its availability, and that is now what has been associated with the outwardly visible and internal signs of age related decline, and others categorized as disease states which can ultimately lead to poor health or death.

Research has shown that augmenting the bioavailability of NAD+ by consuming NAD precursors like NMN and NR can improve and ameliorate the age related declines associated with aging, and possibly extend life itself. It has been shown successful in proving theories in animal models where both the health and lifespan was augmented following the intervention of supplementing the NAD precursors, which include NMN. It has been discovered that a new category of nutraceuticals according to embodiments disclosed herein offer NAD precursors as dietary supplements.

### CD38 and its Effects

Whether it relates to the nicotinamide mononucleotide (NMN), or nicotinamide riboside (NR), their function is roughly the same: to increase NAD levels in the body by orally supplementing precursors like NMN or NR. However, what is now scientifically known is that an extracellular glycoprotein that sit atop many cells in the body called clusters of differentiation 38 (CD38) is now accepted as the leading cause of the NAD decline in our bodies as we age. While the full spectrum of its function is still being realized, CD38 is known to act in a certain regulating capacity for NAD (some tied to our body's immune response), where CD38 accounts for a majority of NAD consumed and degraded through its activities such as protein acetylation in cells throughout the body. Its ubiquitous nature is what makes its activities so consequential to NAD precursors like NMN or NR as well as to NAD+ itself. To which, what is beyond dispute is the fact that its overall effect on NAD+ levels can be directly correlated to most of our disease states tied to our cellular metabolic stasis, which includes heart disease, type II diabetes, and cancer.

Embodiments of the bioactive composition disclosed herein address this age related imbalance through oral supplementation of highly bioactive naturally occurring polyphenols in the flavonoid family known commercially as apigenin and quercetin, as inhibitors of CD38. Both apigenin and quercetin can be found in any number of fruits and vegetables, but parsley and chamomile are the two with the highest concentration of apigenin while red onions has been shown to be especially rich in quercetin. The purified pharmaceutical grade versions of the two flavonoids have shown potent inhibition of CD38 and its catalytic activity as competitive antagonists. This has been shown in studies to result in the significant increase of cellular NAD+ levels resulting in favorable outcomes associated with elevated NAD levels tied to better health.

Applicant has further discovered that the goal of naturally increasing the availability of NAD may be accomplished using a dual action formulation which includes pure apigenin a potent flavonoid shown to fight cancer cells while demonstrating a strong inhibitive effects on CD38, and quercetin a similar yet more popular anti-aging flavonoid known for cellular regeneration, and immune modulating effects, but with less inhibition potency as apigenin, augmenting the synergistic pharmacodynamics paired together as CD38 inhibitors to be formulated along with the NAD precursor NMN according to embodiments disclosed herein. Together, they are thought to interact to bolster the effects of increasing NAD+. While individually, where each component has proven efficacious in raising the serum NAD levels, it is thought that the disclosed apigenin and quercetin work even better synergistically when paired together. Moreover, embodiments further include the dual purposes of reducing the CD38 barriers to the efficacious utility of NAD precursors, and more importantly to raise the bioavailable NAD values. The reasoning is due to current research which indicates that with aging comes the overexpression of CD38 degradation of NAD+ in the body, which is known to be directly tied to age related diseases. Embodiments of the composition are thought to allow for a more efficient elevation of NAD+ without having to megadose in high volume the NAD precursors like NMN and NR, which is further known to be neutralized due to the rate limiting effects of CD38 on NAD levels, including supplementation of precursors. Embodiments disclosed herein thus replace the popular practice among many consumers of NAD precursors to megadose beyond the 1000 mg safety limits (shown in human trials) to compensate for the eventual loss. To which there is little evidence whether this over compensation through megadosing can effectively make up for the deficit caused by CD38 and its effect on the human body. Instead, the embodiments disclosed herein are believed to result in a more efficacious use than NAD precursor supplementation alone. This central tenet is what differentiates embodiments disclosed herein from what is commercially available as NAD precursors.

Embodiments of the composition disclosed herein include a dual action formula which is further enhanced by the addition of an absorption enhancer that is known by its trade name as bioperine or the generic piperine. The absorption enhancer is believed to increase the absorption of active ingredients like apigenin, quercetin, and vitamin B3 (which is related to NMN by its chemical composition) in the small intestine. Moreover, the flavonoid apigenin which is unstable in its purified form, also suffers from poor bioavailability in its natural state as an aglycone. However, apigenin along with quercetin is stabilized and made potent throughout its shelf life (up to 2 years) via glycosidic bonding through the use of dextrin as a polymerizing agent—which is made more bioactive when consumed as an oral supplement. In embodiments, they are encapsulated together in a light protected (cellulosic) capsule with high opacity for added protection and shelf life. Separately, these ingredients have proven efficacy in mouse model studies, however, this unique composition according to embodiments disclosed herein is thought to result in synergistic actions of the actives that comprise the dual-action formula. The values of the active ingredients are as a whole balanced in a safe to use formulation (conforms to safety data from human trials) to reflect the importance of a measured intervention to minimize some of the most deleterious effects of CD38 while giving the body a chance to upregulate its own recycling effort (how more than 90% of NAD is formed). Even a nominally small decrease in CD38 levels can have a significant effect on overall NAD values. Embodiments disclosed herein thus offer an improved opportunity in oral supplementation to enhance the increased availability of NAD in the human body.

### CD38

CD38 refers to cluster of differentiation 38, which is also known as cyclic ADP ribose hydrolase. CD38 is a glycoprotein found on the surface of many immune cells (white blood cells), including CD4+, CD8+, B lymphocytes and natural killer cells. CD38 also functions in cell adhesion, signal transduction and calcium signaling.

In humans, the CD38 protein is encoded by the CD38 gene which is located on chromosome 4. CD38 is a paralog of CD157, which is also located on chromosome 4 (4p15) in humans.

CD38 can function either as a receptor or as an enzyme. As a receptor, CD38 can attach to CD31 on the surface of T cells, thereby activating those cells to produce a variety of cytokines. CD38 is a multifunctional enzyme that catalyzes the synthesis of ADP ribose (ADPR) (97%) and cyclic ADP-ribose (cADPR) (3%) from NAD+. CD38 can be a major regulator of NAD+ levels because 100 molecules of NAD+ is required to generate one molecule of cADPR. When nicotinic acid is present under acidic conditions, CD38 can hydrolyze nicotinamide adenine dinucleotide phosphate (NADP+) to NAADP. These reaction products are essential for the regulation of intracellular Ca²⁺. CD38 occurs not only as an ectoezyme on cell outer surfaces, but also occurs on the inner surface of cell membranes, facing the cytosol performing the same enzymatic functions.

A gradual increase in CD38 has been implicated in the decline of NAD+ with age. Treatment of old mice with a specific CD38 inhibitor, 78c, prevents age-related NAD+ decline. CD38 knockout mice have twice the levels of NAD+ and are resistant to age-associated NAD+ decline, with dramatically increased NAD+ levels in major organs (liver, muscle, brain, and heart). On the other hand, mice overexpressing CD38 exhibit reduced NAD+ and mitochondrial dysfunction.

Macrophages are believed to be primarily responsible for the age-related increase in CD38 expression and NAD+ decline. Macrophages accumulate in visceral fat and other tissues with age, leading to chronic inflammation. Secretions from senescent cells induce high levels of expression of CD38 on macrophages, which becomes the major cause of NAD+ depletion with age.

Decline of NAD+ in the brain with age may be due to increased CD38 on astrocytes and microglia, leading to neuroinflammation and neurodegeneration.

Manipulation of NAD+ metabolism has emerged as a plausible strategy to ameliorate numerous health related issues. In particular, an increase in cellular NAD+ level has beneficial effects, likely because of the activation of sirtuins. It has been reported that CD38 is the primary NAD+ ase in mammals. Moreover, CD38 knockout mice have higher NAD+ levels and are protected against obesity and other issues. CD38 regulates global protein acetylation through changes in NAD+ levels and sirtuin activity.

### CD38 Inhibitors

Both quercetin and apigenin have been shown individually to function as CD38 inhibitors. Applicant has discovered a synergistic effect wherein quercetin in combination with apigenin and the other components of the composition, according to embodiments disclosed herein, shown pharmacological inhibition of CD38 which results in higher intracellular NAD+ levels and the benefits that flow therefrom. Apigenin has also been found to decrease global acetylation as well as the acetylation of p53 and RelA-p65.

Quercetin has been shown to possess antioxidant properties to lessen the risk of disease and quicken aging. Quercetin is a more powerful antioxidant than vitamin C, vitamin E, or beta carotene. Quercetin has also been shown to reduce inflammation caused by stress and injuries and to facilitate the body to heal. Quercetin has been shown to prevent both acute and chronic inflammation, in addition to showing anti-arthritis properties.

Quercetin has also been indicated in the reduction of cancer, having anticancer properties that are thought to prevent the spread of cancerous cells and tumor growth. Quercetin has been indicated as effective to in both the treatment and prevention of prostate cancer. Quercetin has also been utilized in preventing neurological diseases, in particular neurodegenerative diseases such as Alzheimer's or Parkinson's disease. The antioxidant properties of quercetin may help fight free radicals associated with such conditions.

Quercetin has also been indicated as effective to relieve allergy symptoms, acting as an effective antihistamine, it restricts histamine from being released from cells. Anti-allergy properties of quercetin indicate it for treatment of bronchitis and asthma.

Quercetin has also been indicated in preventing infections due to the antibacterial and antiviral properties. In particular, adenovirus, herpes simplex virus, Japanese encephalitis, respiratory syncytial virus, and the like.

Quercetin has also been indicated as useful in reducing the risk of heart disease, improving blood vessel cell health and blood flow through arteries in people with heart disease. Accordingly, quercetin has been shown to lower high blood pressure.

### Increased Bioavailability

Bioperine, also referred to in the art as piperine, is an alkaloid having the general structure:

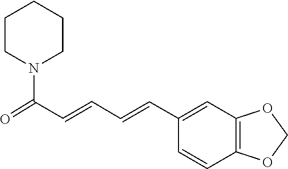 [SMILES: O=C(/C=C/C=C/c1ccc2c(c1)OCO2)N1CCCCC1]

In some embodiments, any liable carbon, nitrogen, and/or oxygen atom may be further substituted as described above to form a derivative thereof.

Bioperine is the alkaloid responsible for the pungency of black pepper and long pepper. Piperine, or mixtures containing piperine, have been shown to increase the bioavailability, blood levels and efficacy of a number of drugs including ingredients of vasaka leaves (Bose, K. G., (1928) Pharmacopeia India, Bose Laboratories, Calcutta), vasicine (Atal et al., Journal of Ethnopharmacology. 229-233 (1981)), sparteine (Atal et al., ibid), sulfadiazine (Atal et al., ibid), rifampicin (Zutshi, U. et al. (1984) Journal of the Association of Physicians of India. 33. 223-224), phenytoin (Bano et al., Planta Medica, 1987, pp. 568-569), pentobarbitone (Majumdar, A. N. et al. (1990), Indian Journal of Experimental Biology. 28. 486-487), theophylline (Bano et al. Eur. J. Clin. Pharmacol. (1991) 41:615-617) and propranolol (ibid).

Piperine is utilized herein to increase the bioavailability of the other components of the composition, e.g., a. In summary, all of these examples clearly illustrate the role of piperine as a drug bioavailability enhancer. The combination of piperine with tested drugs is effective primarily due to higher plasma concentration and a longer stay of the drugs in the body. The reduced dose of highly toxic drugs and their enhanced efficacy is obviously desirable.

The effective bio enhancing dose of piperine for drug compounds varies, but the prior art studies indicate that a dose of approximately 10% (wt/wt) of the active drug could be regarded as an appropriate bio enhancing dose for most drugs.

There are two plausible explanations of the role that piperine may have in drug bioavailability: a) non-specific mechanisms promoting rapid absorption of drugs and nutrients, e.g., increased blood supply to the gastrointestinal tract, decreased hydrochloric acid secretion which prevents breakdown of some drugs, increased emulsifying content of the gut, increased enzymes like gamma-glutamyl transpeptidase which participate in active and passive transport of nutrients to the intestinal cells, and b) non-specific mechanisms inhibiting enzymes participating in biotransformation of drugs, preventing their inactivation and elimination. See: Annamalai, A. R., Manavalan, R. (1990) Effects of ‘Trikatu’ and its individual components and piperine on gastrointestinal tracts: Trikatu—a bioavailable enhancer. Ind. Drugs 27(12); pp. 595-604; Johri, R. K. et al. (1992) Piperine-mediated changes in the permeability of rat intestinal epithelial cells. Bioch. Pharmacol. 43; pp. 1401-1407; Atal, C. K. et al. (1985) Biochemical basis of enhanced drug availability by piperine: Evidence that piperine is a potent inhibitor of drug metabolism. J. Pharmacol. Exp. Therap. 232; pp. 258-262; and Singh, J. et al. (1986) Piperine-mediated inhibition of glucuronidation activity in intestine: evidence that piperine lowers the endogenous UDP-glucuronic acid content. J. Pharmacol. Exp. Therap. 2236; pp. 448-493.

Most drugs co-administered with piperine are probably more bioavailable as a result of both of the mechanisms, i.e., increased absorption from the gut and the slow down of biotransformation, inactivation and elimination from the system. The latter mechanism is probably the most important in sustaining the elevated blood levels of the drug, and making it more bioavailable to the tissue. Although a rapid absorption to the blood stream may account for increased blood levels of the drug, it is the inhibition of drug bio transforming enzymes with piperine that makes a drug stay in the body longer, in higher quantities, which makes it more effective.

Based on available literature data, it seems that piperine in a daily dose of at least 20 mg per person operates through inhibiting enzymes that would otherwise bio transform and speed up elimination of many drugs (Zutshi, U. et al. (1989) A process for the preparation of pharmaceutical combination with enhanced activity for treatment of tuberculosis and leprosy. Indian Patent No. 1231/Del/89; Zutshi et al. (1984) Influence of piperine on rifampicin blood levels in patients of pulmonary tuberculosis. J. Assoc. Phys. Ind. 33; pp. 223-224; Ban, C. K. et al. (1991) The effect of piperine on the bioavailability and pharmacokinetics of propranolol and theophylline in healthy volunteers. European J. Clin. Pharm. 41; pp. 615-618 and Bano, G. et al. (1978) The effect of piperine on the pharmacokinetics of phenytoin in healthy volunteers. Planta Medica 53; pp. 568-570).

Interestingly, the dose of piperine that inhibits the bio transforming enzymes operates regardless of whether it is administered concurrently with the drug. This point can be illustrated by experiments with theophylline and phenytoin, where 20 mg of piperine was administered for seven days prior to the administration of either drug [Ban, C. K. et al. (1991) The effect of piperine on the bioavailability and pharmacokinetics of propranolol and theophylline in healthy volunteers. European J. Clin. Pharm. 41; pp. 615-618 and Bano, G. et al. (1978) The effect of piperine on the pharmacokinetics of phenytoin in healthy volunteers. Planta Medica 53; pp. 568-570]. Since that regimen resulted in increased blood levels of the administered drugs, and dramatically prolonged the elimination time, the plausible explanation is that the prior administration of piperine inhibited drug bio transforming enzymes. In fact, this seems to be the only explanation for the increased bioavailability, since piperine administered separately from the drug could not possibly affect gastrointestinal events leading to its rapid absorption.

Another interesting observation is that doses of piperine below what is considered effective in inhibiting the bio transforming enzymes, may still be sufficient to enhance the rapid absorption of a drug from the gut. This phenomenon can be illustrated by the co-administration of piperine with the anti-hypertensive drug propranolol (Zutshi, U. et al. (1989) A process for the preparation of pharmaceutical combination with enhanced activity for treatment of tuberculosis and leprosy. Indian Patent No. 1231/Del/89).

Propranolol when administered with piperine showed a significant increase in blood levels. The maximum blood concentration of the drug increased two fold with piperine. Importantly, despite dramatically improving the bioavailability of propranolol, piperine, as used in a 3 mg dose, did not affect the elimination rate of the drug. In an experimental design distinct from previous studies, the anti-asthmatic drug theophylline and the anti-epileptic drug phenytoin were tested (Ban, C. K. et al. (1991) The effect of piperine on the bioavailability and pharmacokinetics of propranolol and theophylline in healthy volunteers. European J. Clin. Pharm. 41; pp. 615-618 and Bano, G. et al. (1978) The effect of piperine on the pharmacokinetics of phenytoin in healthy volunteers. Planta Medica 53; pp. 568-570). The study was done on six healthy volunteers. The participants were pretreated with 20 mg of piperine daily for seven days before receiving 150 mg of theophylline or 300 mg of phenytoin.

The maximum concentration of theophylline was 1.5 times higher in subjects pretreated with piperine. Importantly, the elimination rate of the drug was significantly slowed down with piperine pretreatment.

Phenytoin blood concentration rose more rapidly in the group pretreated with piperine than in the group receiving the drug alone. The pretreated group attained maximum concentration of the drug in shorter time and in significantly higher concentrations. The pretreatment with piperine resulted in significantly slower elimination of the drug. The prior art discussed above clearly illustrates the role of piperine as a bioavailability enhancer, and the importance of its effective dose on the overall mechanism of enhanced bioavailability.

In the case of propranolol, the co-administration with only 3 mg of piperine resulted in doubling its blood levels, but without slowing down the drug elimination rate. Thus, it may be inferred that, in a small dose, piperine may not inhibit the bio transforming enzymes or affect the elimination rate of a drug. Rather, it may operate through enhancement of gastrointestinal events leading to rapid absorption mechanisms.

### NAD Precursor

Nicotinamide mononucleotide (NMN) has the general structure

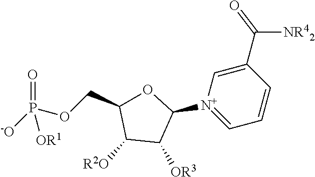

wherein each of R¹, R², R³, and R⁴, are hydrogen. In embodiments, the nicotinamide mononucleotide is present as a derivative, which for purposes herein is defined as having the above structure, wherein one or more of R¹, R², R³, and R^{4′}, are substituted with a C₁-C₄₀ hydrocarbyl radical, a substituted hydrocarbyl radical, a functional group comprising elements from Group 13-17 of the periodic table of the elements.

NMN has been shown to treat mitochondrial disease and other disorders characterized by deficiency in NAD+ levels and NAD+ redox imbalance. Nicotinamide mononucleotide (NMN) has been shown to extend the lifespan of subjects by normalizing NAD+ redox imbalance and lowering accumulation of toxins in various muscles without affecting the brain. NMN has also been shown to up-regulate alphaketoglutarate (KG) levels in muscle.

### Other Components

In embodiments, the composition may further include one or more of epigallocatechin gallate (EGCG), baicalin, hesperidin, myricetin, luteolin, Vitamin B3, zinc salts or complexes, and/or Vitamin D. In embodiments, the composition may further comprise from about 50 to 150 mg of baicalin or a derivative thereof; from about 50 to 150 mg of hesperidin or a derivative thereof; from about 35 to 150 mg of myricetin or a derivative thereof; from about 25 to 75 mg of luteolin or a derivative thereof; from about 10 to 50 mg of Vitamin B3 or a complex thereof; from about 10 to 50 mg of zinc as a salt or complex, based on the metal content present; from about 2000 IU to 7500 IU of Vitamin D or a complex thereof; or a combination thereof.

Epigallocatechin gallate (EGCG), which refers to a compound having the following general formula:

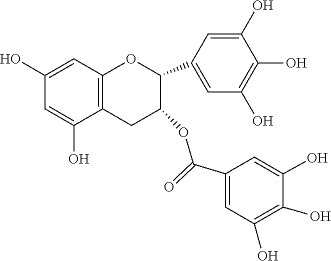

In embodiments, one or more of the phenolic hydrogens may be substituted with one or more functional groups as disclosed herein. EGCG is a polyphenol having potential to affect human health and disease. EGCG is known to reduce inflammation, aid weight loss, and help prevent heart and brain disease.

Hesperidin refers to a flavanone glycoside found in citrus fruits, and having the general structure:

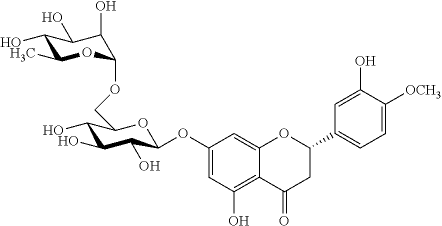

In glycone form it is called hesperetin. In embodiments, the hesperidin is present as a derivative, which for purposes herein is defined as having the above structure, wherein one or more of the hydroxyl hydrogens are substituted with a C₁-C₄₀ hydrocarbyl radical, a substituted hydrocarbyl radical, a functional group comprising elements from Group 13-17 of the periodic table of the elements.

Hesperitin is known to mediate the actions of hesperidin in the body, and since hesperidin needs to progress to the colon to be ‘released’ by intestinal bacteria it acts as a time-release for hesperitin; one serving of hesperidin seems to increased blood levels for over the course of a day or so when consumed in this manner. Orally ingested hesperidin is known to promote blood flow and impact other health related systems in mammals.

Luteolin is a flavone, having the following structure:

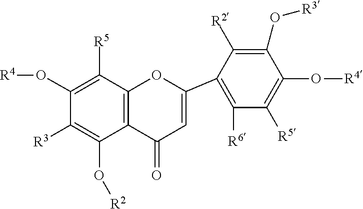

wherein each of R², R³, R⁴, R⁵, R^{2′}, R^{3′}, R^{4′}, R^{5′}, and R^{6′} are hydrogen. In embodiments, the luteolin is present as a derivative, which for purposes herein is defined as having the above structure, wherein one or more of R¹, R², R³, R⁴, R⁵, R^{2′}, R^{3′}, R^{4′}, R^{5′}, and R^{6′} are substituted with a C₁-C₄₀ hydrocarbyl radical, a substituted hydrocarbyl radical, a functional group comprising elements from Group 13-17 of the periodic table of the elements, or wherein two or more of R¹, R², R³, R⁴, R⁵, R^{2′}, R^{3′}, R^{4′}, R^{5′}, and R^{6′} independently join together to form a C₄ to C₆₂ cyclic or polycyclic ring structure.

Luteolin is known to form complexes with the four bases of DNA suggesting possible anti-viral properties.

Baicalin is a flavone glycoside having the general formula:

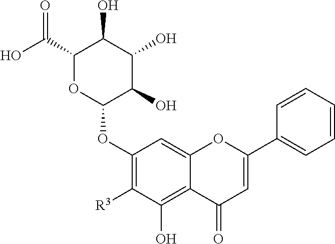

In embodiments, the baicalin is present as a derivative, which for purposes herein is defined as having the above structure, wherein one or more of the hydroxyl hydrogens are substituted with a C₁-C₄₀ hydrocarbyl radical, a substituted hydrocarbyl radical, a functional group comprising elements from Group 13-17 of the periodic table of the elements.

Baicalin, along with its aglycone baicalein, is a positive allosteric modulator of the benzodiazepine site and/or a non-benzodiazepine site of the GABAA receptor. Baicalin is known to produce anxiolytic effects without sedative or myorelaxant effects, and is thought to act with other flavonoids to underlie various anxiolytic effects. Baicalin is also a known prolyl endopeptidase inhibitor having activity that induces apoptosis in pancreatic cancer cells among other effects.

Myricetin is a flavone, having the following structure:

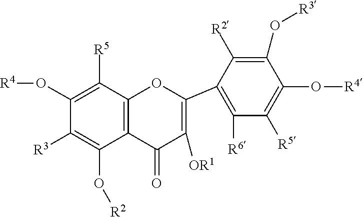

wherein each of R¹, R², R³, R⁴, R⁵, R^{2′}, R^{3′}, R^{4′}, R^{5′}, and R^{6′} are hydrogen. In embodiments, the myricetin is present as a derivative, which for purposes herein is defined as having the above structure, wherein one or more of R¹, R², R³, R⁴, R⁵, R^{2′}, R^{3′}, R^{4′}, R^{5′}, and R^{6′} are substituted with a C₁-C₄₀ hydrocarbyl radical, a substituted hydrocarbyl radical, a functional group comprising elements from Group 13-17 of the periodic table of the elements, or wherein two or more of R¹, R², R³, R⁴, R⁵, R^{2′}, R^{3′}, R^{4′}, R^{5′}, and R^{6′} independently join together to form a C₄ to C₆₂ cyclic or polycyclic ring structure.

Myricetin is a member of the flavonoid class of polyphenolic compounds, with antioxidant properties and is structurally similar to fisetin, luteolin, and quercetin and is reported to have many of the same functions as these other members of the flavonol class of flavonoids.

Vitamin B3 refers to one or more of the three forms: nicotinamide (niacinamide), niacin (nicotinic acid), and nicotinamide riboside. Each may be present alone, or in a group typically referred to as vitamin B3 complex.

Vitamin D refers to the group of fat-soluble secosteroids known to be responsible for increasing intestinal absorption of calcium, magnesium, and phosphate, and multiple other biological effects. Vitamin D may be present as vitamin D3, also known as cholecalciferol, and/or as vitamin D2, known as ergocalciferol as is standard in the art.

In embodiments, zinc is present as a salt, clathrate, or coordinated with one or more ligands. Suitable forms of zinc for purposes herein include zinc gluconate, zinc acetate, zinc sulfate, zinc picolinate, zinc orotate, and/or zinc citrate.

In embodiments, a composition comprises a biologically active amount of nicotinamide mononucleotide or a derivative thereof, apigenin or a derivative thereof, quercetin or a derivative thereof, and bioperine or a derivative thereof. In some embodiments, the composition further includes baicalin, hesperidin, myricetin, EGCG, luteolin, Vitamin B3, zinc (as a salt), Vitamin D, or a combination thereof.

### Compositions

In embodiments, a composition comprises a biologically active amount of nicotinamide mononucleotide or a derivative thereof, apigenin or a derivative thereof, quercetin or a derivative thereof, and piperine or a derivative thereof. In some embodiments, the weight to weight ratio of apigenin to nicotinamide mononucleotide is from about 0.4 to about 0.8, based on the total amount of apigenin and nicotinamide mononucleotide present, and/or the weight to weight ratio of quercetin to nicotinamide mononucleotide is from about 0.2 to about 0.6, based on the total amount of quercetin and nicotinamide mononucleotide present, and/or the weight to weight ratio of piperine to nicotinamide mononucleotide is from about 0.02 to about 0.06, based on the total amount of piperine and nicotinamide mononucleotide present.

In embodiments, the composition further comprises: from about 50 to 150 mg of baicalin or a derivative thereof; from about 50 to 150 mg of hesperidin or a derivative thereof; from about 35 to 150 mg of myricetin or a derivative thereof; from about 25 to 75 mg of luteolin or a derivative thereof; from about 10 to 50 mg of Vitamin B3 or a complex thereof; from about 10 to 50 mg of zinc as a salt or complex, based on the metal content present; from about 2000 IU to 7500 IU of Vitamin D or a complex thereof; or a combination thereof.

In embodiments, the composition comprises from about 50 mg to 350 mg of nicotinamide mononucleotide or a derivative thereof. In embodiments, the composition comprises from about 10 to 200 mg of apigenin or a derivative thereof. In embodiments, the composition comprises from about 10 to 200 mg of quercetin or a derivative thereof. In embodiments, the composition comprises from about 1 to 20 mg of bioperine or a derivative thereof.

In embodiments of the composition, the nicotinamide mononucleotide, the apigenin, the quercetin, and/or the bioperine are present as one or more glycoside derivatives.

In embodiments, the composition further comprises a carrier, diluent, preservative, or a combination thereof comprising one or more of glucose, fructose, sucrose, maltose, yellow dextrin, white dextrin, aerosol, cellulose, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.

In embodiments, the composition is in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.

In embodiments of the composition, the nicotinamide mononucleotide or the derivative thereof, the apigenin or the derivative thereof, the quercetin or the derivative thereof, and/or the bioperine or the derivative thereof are extracts, fractions, compounds, phytochemicals, and/or powders derived from plants.

In embodiments, the composition is in the form of an oral dosage suitable for administration to a mammal.

A method to treat a physiological condition of a mammal comprises the steps of providing a composition according to one or more embodiment disclosed herein; and administering a biologically effective dosage to the mammal.

In embodiments, the physiological condition comprises: diabetes, hyperglycemia, hypolipidemia; obesity, hypertension, platelet aggregation; viral infection, atherosclerosis, inflammation, auto-immune disfunction, or a combination thereof. In some embodiments, the physiological condition comprises a viral infection.

In some embodiments, the composition consisting essentially of a biologically active amount of nicotinamide mononucleotide or a derivative thereof, apigenin or a derivative thereof, quercetin or a derivative thereof, and piperine or a derivative thereof. In such an embodiment, the composition includes a carrier, a diluent, a preservative, cellulose, dextrin, magnesium stearate, or a combination thereof in the form of a tablet, soft capsule, hard capsule, a pill, or a combination thereof. In such an embodiment, the composition has from about 50 mg to 350 mg of nicotinamide mononucleotide or a derivative thereof, from about 10 mg to 200 mg of apigenin or a derivative thereof, from about 10 mg to 200 mg of quercetin or a derivative thereof, and from about 1 mg to 20 mg of piperine or a derivative thereof.

In some embodiments, the weight to weight ratio of apigenin to nicotinamide mononucleotide is from 1:99 to 99:1, based on the total amount of apigenin and nicotinamide mononucleotide present. In embodiments, the weight to weight ratio of apigenin to nicotinamide mononucleotide is from about 0.4 to about 0.8, based on the total amount of apigenin and nicotinamide mononucleotide present. In embodiments, the weight to weight ratio of apigenin to nicotinamide mononucleotide is greater than or equal to about 0.4, or greater than or equal to about 0.5, or greater than or equal to about 0.6, or greater than or equal to about 0.7, based on the total amount of apigenin and nicotinamide mononucleotide present. In embodiments, the weight to weight ratio of apigenin to nicotinamide mononucleotide is less than or equal to about 0.8, or less than or equal to about 0.7, or less than or equal to about 0.6, or less than or equal to about 0.5, based on the total amount of apigenin and nicotinamide mononucleotide present.

In embodiments, the weight to weight ratio of quercetin to nicotinamide mononucleotide is from 1:99 to 99:1, based on the total amount of quercetin and nicotinamide mononucleotide present. In embodiments, the weight to weight ratio of quercetin to nicotinamide mononucleotide is from about 0.2 to about 0.6, based on the total amount of quercetin and nicotinamide mononucleotide present. In embodiments, the weight to weight ratio of quercetin to nicotinamide mononucleotide is greater than or equal to about 0.2, or greater than or equal to about 0.3, or greater than or equal to about 0.4, or greater than or equal to about 0.5, based on the total amount of quercetin and nicotinamide mononucleotide present. In embodiments, the weight to weight ratio of quercetin to nicotinamide mononucleotide is less than or equal to about 0.6, or less than or equal to about 0.5, or less than or equal to about 0.4, or less than or equal to about 0.3, based on the total amount of quercetin and nicotinamide mononucleotide present.

In embodiments, the weight to weight ratio of piperine (bioperine) to nicotinamide mononucleotide is from about 1:99 to 99:1 based on the total amount of bioperine and nicotinamide mononucleotide present. In embodiments, the weight to weight ratio of piperine (bioperine) to nicotinamide mononucleotide is from about 0.02 to about 0.06, based on the total amount of bioperine and nicotinamide mononucleotide present. In embodiments, the weight to weight ratio of piperine to nicotinamide mononucleotide is greater than or equal to about 0.02, or greater than or equal to about 0.03, or greater than or equal to about 0.04, or greater than or equal to about 0.05, based on the total amount of piperine and nicotinamide mononucleotide present. In embodiments, the weight to weight ratio of piperine to nicotinamide mononucleotide is less than or equal to about 0.06, or less than or equal to about 0.05, or less than or equal to about 0.04, or less than or equal to about 0.03, based on the total amount of piperine and nicotinamide mononucleotide present.

In embodiments, the composition comprises from about 10 mg to about 1000 mg of nicotinamide mononucleotide or a derivative thereof. In embodiments, the composition comprises greater than or equal to about 50 mg, or greater than or equal to about 75 mg, or greater than or equal to about 100 mg, or greater than or equal to about 150 mg, or greater than or equal to about 200 mg, or greater than or equal to about 250 mg, or greater than or equal to about 300 mg, or greater than or equal to about 350 mg, or greater than or equal to about 400 mg, or greater than or equal to about 500 mg of nicotinamide mononucleotide or a derivative thereof.

In embodiments, the composition comprises less than or equal to about 500 mg, or less than or equal to about 400 mg, or less than or equal to about 350 mg, or less than or equal to about 300 mg, or less than or equal to about 250 mg, or less than or equal to about 200 mg, or less than or equal to about 150 mg, or less than or equal to about 100 mg, or less than or equal to about 75 mg, or less than or equal to about 50 mg of nicotinamide mononucleotide or a derivative thereof.

In embodiments, the composition comprises from about 10 mg to about 500 mg of apigenin or a derivative thereof. In embodiments, the composition comprises greater than or equal to about 20 mg, or greater than or equal to about 30 mg, or greater than or equal to about 50 mg, or greater than or equal to about 75 mg, or greater than or equal to about 100 mg, or greater than or equal to about 150 mg, or greater than or equal to about 200 mg, or greater than or equal to about 300 mg, or greater than or equal to about 400 mg, or greater than or equal to about 450 mg of apigenin or a derivative thereof.

In embodiments, the composition comprises less than or equal to about 500 mg, or less than or equal to about 400 mg, or less than or equal to about 300 mg, or less than or equal to about 200 mg, or less than or equal to about 150 mg, or less than or equal to about 75 mg, or less than or equal to about 50 mg, or less than or equal to about 40 mg, or less than or equal to about 30 mg, or less than or equal to about 25 mg of apigenin or a derivative thereof.

In embodiments, the composition comprises from about 10 mg to about 500 mg of quercetin or a derivative thereof. In embodiments, the composition comprises greater than or equal to about 20 mg, or greater than or equal to about 30 mg, or greater than or equal to about 50 mg, or greater than or equal to about 75 mg, or greater than or equal to about 100 mg, or greater than or equal to about 150 mg, or greater than or equal to about 200 mg, or greater than or equal to about 300 mg, or greater than or equal to about 400 mg, or greater than or equal to about 450 mg of quercetin or a derivative thereof.

In embodiments, the composition comprises less than or equal to about 500 mg, or less than or equal to about 400 mg, or less than or equal to about 300 mg, or less than or equal to about 200 mg, or less than or equal to about 150 mg, or less than or equal to about 75 mg, or less than or equal to about 50 mg, or less than or equal to about 40 mg, or less than or equal to about 30 mg, or less than or equal to about 25 mg of quercetin or a derivative thereof.

In embodiments, the composition comprises from about 1 to 50 mg of bioperine or a derivative thereof. In embodiments, the composition comprises greater than or equal to about 2 mg, or greater than or equal to about 3 mg, or greater than or equal to about 5 mg, or greater than or equal to about 10 mg, or greater than or equal to about 20 mg, or greater than or equal to about 25 mg, or greater than or equal to about 30 mg, or greater than or equal to about 35 mg, or greater than or equal to about 40 mg, or greater than or equal to about 45 mg of bioperine or a derivative thereof.

In embodiments, the composition comprises less than or equal to about 50 mg, or less than or equal to about 45 mg, or less than or equal to about 35 mg, or less than or equal to about 30 mg, or less than or equal to about 25 mg, or less than or equal to about 20 mg, or less than or equal to about 15 mg, or less than or equal to about 10 mg, or less than or equal to about 5 mg, or less than or equal to about 3 mg of bioperine or a derivative thereof.

In embodiments, the nicotinamide mononucleotide, the apigenin, the quercetin, and/or the bioperine are present as one or more glycoside derivatives. In embodiments, the composition further comprises a carrier, diluent, preservative, or a combination thereof comprising one or more of glucose, fructose, sucrose, maltose, yellow dextrin, white dextrin, aerosol, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.

In embodiments, the composition is in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.

In embodiments, the nicotinamide mononucleotide or the derivative thereof, the apigenin or the derivative thereof, the quercetin or the derivative thereof, and/or the bioperine or the derivative thereof are extracts, fractions, compounds, phytochemicals, and/or powders derived from plants.

In embodiments, the composition is in the form of an oral dosage suitable for administration to a mammal.

### Other Uses of the Composition

Viral infections are known to be difficult to treat with the pharmaceutical anti-viral compounds currently available for treatment of such. Applicant has discovered another option. Compositions according to embodiments disclosed herein assist the body through both direct and indirect intervention with naturally occurring compounds that can help it fight off the viral infection by helping the body to address its vulnerability while empowering the body's innate immune response, and allow it to stay in charge of the fight until other treatment become available. At the very least this option buys time until other treatment becomes available, even if it is unable to help in the immediate protection and removal of the virus itself. The compounds in question have had years of research to prove its use and efficacy. Where it may have fallen short has more to do with its application and formulation than its validation as a useful antiviral compound.

Embodiments of the composition provide a potent combination of polyphenols as are known to be available in nature. It is this subgroup called flavonoids which has shown great deal of promise as a prophylactic as well as a treatment, and where useful as a supporting role for which it can be useful in interventionary medicine. In embodiments, the flavonoids utilized in the compositions herein are sourced from fruits, vegetables, nuts, seeds, flowers and/or the like. They hold a broad range of potential for a variety of curative biological and pharmacological activities, such as antimicrobial, anti-inflammatory, anticarcinogenic, antimicrobial, anticancer, and antiviral properties.

In embodiments, the flavonoids including flavones, flavonols isoflavones, anthocyanins, and the like act in a synergistic relationship to provide self-health initiatives. Numerous studies conducted in the last twenty years have validated flavonoids as curative and efficacious in their natural compound forms. They have become the mainstay in their supplement form as many consumers have found good utility for its use, whether due to its anecdotal efficacy or because of their belief that alternative health can offer safer if not better measures to maintain wellness.

It is thought compositions according to embodiments disclosed herein possess high potency as anti-mutagenic, anticancer, and virucidal medicines of first resort. Applicant has discovered that embodiments of the compositions disclosed herein offer some of nature's best protection against viral infections through immune optimization and modulation.

It is in addressing the need to offer an easily affordable self-health option for increasing one's chance of surviving viral infections, embodiments of the compositions disclosed herein provide a low risk antiviral with the potential at the very least to mitigate some if not substantial amount of the horrendous health effects of various viruses, including coronaviruses. This approach takes the all-natural flavonoid based to confer prophylactic protection and mitigation against the severity of the viral infection.

Using proof of principle provided by institutional meta-data directed to evidence and substance to its utility, Applicant has discovered a prophylactic without the side-effect that accompany most drugs. Especially the at risk population with virtually no protection present against it.

The basis for the instant proof of principle is grounded in a wealth of data directed to each of the component's biological properties and pharmacokinetics as it pertains to its use (both in animal and human studies). Whereas what is now know about using the flavonoids as medicines in study setting has often invoked by the study participants and solicited for further studies and analysis to discover its true potential as medicine for human consumption. Applicant has taken that concept further by formulating a particular group of flavonoid compounds having synergistic properties when paired in the right combinations as evidenced by embodiments disclosed herein.

Quercetin (flavone) confer greater efficacy in prophylaxis protection as well as a treatment to various disease states, like cancer, cardio-vascular diseases, and of course viral infections, far more so than as a standalone compound used as dietary supplements. Applicant has discovered that Quercetin is far more useful in potential if it is paired and formulated in a way that can make full use of its potential according to computerized modeling and murine mouse models.

Applicant has discovered that the true potential of Quercetin as an antiviral can be realized only when the poor bioavailability of the material is overcome. Whether it is due to its actions following gastric absorption or being made available through targeted pathways, Quercetin is often relegated into organ specific holding pens (kidneys, liver, GI tract) or stuck as cofactors in blood cells to ameliorate its shortcomings. Applicant has discovered that a way to enhance Quercetin permeation within the human body with its anti-inflammatory, and immunologically optimizing capabilities. It has been discovered that when Quercetin is paired with Apigenin, and optionally Luteolin, these materials work in tandem to allow a more robust and broader application of the synergistic pairings of these flavones and flavonoids by formulating them in a way which will allow their best attributes to present itself, much like the cocktail approach which has demonstrated to be more successful with incurable diseases like AIDS.

In embodiments, the compositions provide a broad spectrum high potency polyphenol formula of synergistic flavonoids grouped with other efficacy enhancing flavones, and EGCG (active ingredient to green tea), with supportive supplement B3/niacin which confers upper respiratory protection with Zinc gluconate a naturally antiviral element in the periodic table. This is aided by the absorption enhancement it receives from Piperine (from black peppers), which can allow up to 200% more of the flavonoids to be absorbed.

Embodiments of the composition comprise a cocktail of flavonoid compounds which are coupled with other performance enhancer to improve efficacy via process known as protease inhibition which debilitates the enzymatic process required for the virus to replicate it's genetic codes within the host cell and it's homeostatic attributes it harnesses in infecting the body. The utility of the flavonoids is tied to its innate enzymatic ability to bind to the RNA replication enzymes and resist the viral maturation and development of its viral protein codes in the cells of our body.

The instant compositions comprise components selected to have bioactive antiviral broad spectrum capabilities, which work together in sync to deliver the components to the target of impact. In addition, the flavonoids of the presently claimed invention are formulated with synergistic and safe active ingredients to enhance efficacy by both inhibition and attrition of the viral load with minimal cytotoxicity, based on a well-established principle of both eastern medicine and western naturopathic remedies, where strength of the dose should be modified to the severity of the disease and its symptoms, as well as the opportunistic potential to heal the patient.

The synergistic properties of the various components provide effect by utilizing different points of access to exploit viral vulnerabilities, as opposed to a single treatment options known in the art. In doing so, the instant composition confers the means to arrest its cell to cell advancement. This is predicated on the composition's ability to inhibit and suppress protease and polymerase mediated activity by which it replicates with great efficiency by using the hosts cells. It is in addressing this threat that the titular strategy in how to best defeat a single strand RNA virus with numerous nucleotide components which works to replicate itself using the hosts DNA and much of its transcriptional enzymes it harnesses, lies with series of attacks using such multifaceted compounds to stress the viral load into submission in stages as well as through a coordinated and synchronous effort.

In view of the numerous polyprotein target sections in various viruses, e.g., 3CLPro, PLPro, and RdRp, the multifaceted approach of the instant compositions and methods allow for a greater opportunity to find and exploit susceptible targets of attack. Success of which is especially of vital importance when most if not all the antiviral medication face a high cost for failing, when it can lead to drug resistance or worse a more potent strain.

Embodiments of the composition confer preventative respiratory protection against severe lung damage, which is a baseline necessity where prophylaxis against some viruses is concerned. The instant composition includes powerful flavones like Apigenin, renowned for its potent anticancer, anti-mutagenic, and anti-inflammatory activities; along with other opportunistic inhibitors of the protein to protein viral docking mechanism based on the newest findings; as well as Quercetin a well-researched antiviral all taking turns to neutralizing the S shaped furin spikes to prevent its attachment to the viral entry point, together and separately. It is by competing with and preventing the docking and binding of the virus with the ACE2 receptor sites along with its use of the entry enabler enzyme TMPRSS2 which may offer one the most opportunistic way in which to prevent a virus from infecting the host.

The efficacy is based on results that have been well documented in studies with numerous viruses such as E71 virus/Coxsackie, H1n1, H1N5, Epstein-Barr, Hepatitis C, Chikungunya, Ebola, Zika, and the like, in which the selected flavonoids were used as the primary source of antiviral treatment, and which proved to be successful according to the WHO parameters for success.

In addition, should a viral infection take hold, Quercetin, Apigenin, optionally coupled with Luteoline and/or Hesperidin, is well positioned to offer pre and post infection antiviral mitigation, if not arrest by multifaceted means of inhibiting its maturation and reproduction process. This is especially relevant as it relates to its inhibition of protease enzymes which has been cited from studies done in the wake of SARS Cov1 epidemic that showed this approach to be effective in inhibiting the protease enzyme 3CLpro, which is vital to the SAR Cov1 virus maturation and replication process.

As such, the instant composition is well suited to attack a key component of viral genome proteomic replication with the goal of inhibition, due to the likelihood that SARS Cov2 virus may have more proteomic and enzymatic depth allowing it enhanced replication capabilities.

The flavonoids supported by the other components having well known antioxidant properties further provide a potent antiviral, which collectively do more together to stop the spread of a virus. Together the components act like a powerful cocktail of drugs with its various unique properties that inhibits the virus, but with even more the virucidal potential in its combined formulations which bears a strong correlation with similar approaches proven successful in stopping viral progression. Combined, they can work more efficaciously together than as a sum of single compounds, both in terms of its inhibition potential but also for its anti-inflammatory effects by suppressing inflammatory agents like NO, and COX2 enzymes, along with rate limiting modifying effects on IL-6, IL-8, IL-10, IL1ra (interleukin), and TNF (tumor necrosis factor), all crucial to moderate the lethal immune response known as the cytokine storm. It is the differentiating features of this formula which may prove vital to the key towards improving the chance to surviving a virus attack and mitigating its damage to the body.

In other embodiments, the composition may include zinc in one or more forms, which is one of the most potent antiviral minerals known in nature. Zinc ions have often proven toxic to most viruses even in small amounts in an infected cell. In view of the poor distribution and perfusion of dietary zinc, it needs helpers in the way of compounds like Quercetin and the like to act as zinc ionospheres that helps the zinc enter cells more efficiently for virucidal efficacy as it assists in impairing and degrading the viral RNA synthesis. It is this synergistic bioavailability process that embodiments of the instant composition enable, along with enhanced absorption by Piperine. The compositions disclosed herein possess natural antiviral properties and in combination, have the potential to play a role for those who are infected with the virus by multifaceted approach to shutting down the complex viral transcription process.

In an alternative embodiment, a composition comprises a biologically active amount of trans-resveratrol or a derivative thereof, apigenin or a derivative thereof, quercetin or a derivative thereof, and bioperine or a derivative thereof.

Resveratrol has been shown effective to lower blood pressure due to antioxidant properties. Resveratrol may accomplish this blood-pressure-lowering effect by helping to produce more nitric oxide, which causes blood vessels to relax.

Resveratrol has also been shown effective to decrease LDL cholesterol oxidation, as well as lengthening the lifespan in mammals. Resveratrol has also been shown effective to protect the brain due to the antioxidant and anti-inflammatory activity of resveratrol. It has also been indicated in interfering with protein fragments called beta-amyloids, which are crucial to forming the plaques that are a hallmark of Alzheimer's disease.

Resveratrol has also been shown effective to increase insulin sensitivity and thus provide several benefits for treatment of diabetes, as well as in treatment of arthritis and joint pain by preventing cartilage from breaking down.

Resveratrol has also been indicated to suppress cancer cells by preventing cancer cells from replicating and spreading through changes in gene expression.

In some embodiments, the composition further comprises a biologically active amount of one or more of hesperidin, luteolin, EGCG, Vitamin B3, Zinc, baicalin, myricetin, or a combination thereof. In some embodiments, the zinc is present as a salt, preferably as zinc gluconate.

### Dosage Amounts

In embodiments, when taken in combination according to one or more embodiments disclosed herein, biologically active amounts of apigenin for an adult human include a daily dosage greater than or equal to about 100 mg in 24 hrs, or greater than or equal to about 150 mg in 24 hrs, or greater than or equal to about 300 mg in 24 hrs, or greater than or equal to about 450 mg in 24 hrs, and less than or equal to about 1000 mg in 24 hrs.

In embodiments, when taken in combination according to one or more embodiments disclosed herein, biologically active amounts of trans-resveratrol for an adult human include a daily dosage greater than or equal to about 200 mg in 24 hrs, or greater than or equal to about 250 mg in 24 hrs, or greater than or equal to about 500 mg in 24 hrs, or greater than or equal to about 750 mg in 24 hrs, and less than or equal to about 1000 mg in 24 hrs.

In embodiments, when taken in combination according to one or more embodiments disclosed herein, biologically active amounts of nicotinamide mononucleotide for an adult human include a daily dosage greater than or equal to about 200 mg in 24 hrs, or greater than or equal to about 250 mg in 24 hrs, or greater than or equal to about 500 mg in 24 hrs, or greater than or equal to about 750 mg in 24 hrs, and less than or equal to about 1000 mg in 24 hrs.

In embodiments, when taken in combination according to one or more embodiments disclosed herein, biologically active amounts of quercetin for an adult human include a daily dosage greater than or equal to about 50 mg in 24 hrs, or greater than or equal to about 100 mg in 24 hrs, or greater than or equal to about 200 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of bioperine for an adult human include a daily dosage greater than or equal to about 1 mg in 24 hrs, or greater than or equal to about 5 mg in 24 hrs, or greater than or equal to about 10 mg in 24 hrs, or greater than or equal to about 15 mg in 24 hrs, and less than or equal to about 100 mg in 24 hrs.

In embodiments, when taken in combination according to one or more embodiments disclosed herein, biologically active amounts of baicalin for an adult human include a daily dosage greater than or equal to about 50 mg in 24 hrs, or greater than or equal to about 100 mg in 24 hrs, or greater than or equal to about 200 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of hesperidin for an adult human include a daily dosage greater than or equal to about 50 mg in 24 hrs, or greater than or equal to about 100 mg in 24 hrs, or greater than or equal to about 200 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of myricetin for an adult human include a daily dosage greater than or equal to about 35 mg in 24 hrs, or greater than or equal to about 75 mg in 24 hrs, or greater than or equal to about 150 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of EGCG for an adult human include a daily dosage greater than or equal to about 35 mg in 24 hrs, or greater than or equal to about 75 mg in 24 hrs, or greater than or equal to about 150 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of luteolin for an adult human include a daily dosage greater than or equal to about 25 mg in 24 hrs, or greater than or equal to about 50 mg in 24 hrs, or greater than or equal to about 100 mg in 24 hrs, or greater than or equal to about 300 mg in 24 hrs, and less than or equal to about 1000 mg in 24 hrs.

In embodiments, when taken in combination according to one or more embodiments disclosed herein, biologically active amounts of Vitamin B3 for an adult human include a daily dosage greater than or equal to about 10 mg in 24 hrs, or greater than or equal to about 20 mg in 24 hrs, or greater than or equal to about 40 mg in 24 hrs, or greater than or equal to about 100 mg in 24 hrs, and less than or equal to about 500 mg in 24 hrs.

In embodiments, when taken in combination according to one or more embodiments disclosed herein, biologically active amounts of zinc, in terms of the equivalent zinc metal for an adult human include a daily dosage greater than or equal to about 10 mg in 24 hrs, or greater than or equal to about 20 mg in 24 hrs, or greater than or equal to about 40 mg in 24 hrs, or greater than or equal to about 100 mg in 24 hrs, and less than or equal to about 500 mg in 24 hrs.

In embodiments, when taken in combination according to one or more embodiments disclosed herein, biologically active amounts of Vitamin D for an adult human include a daily dosage greater than or equal to about 1250 IU in 24 hrs, or greater than or equal to about 2500 IU in 24 hrs, or greater than or equal to about 5000 IU in 24 hrs, and less than or equal to about 10,000 IU in 24 hrs.

In embodiments, the weight to weight ratio of apigenin to trans-resveratrol is from about 0.4 to about 0.8, based on the total amount of apigenin and trans-resveratrol present. In embodiments, the weight to weight ratio of quercetin to trans-resveratrol is from about 0.2 to about 0.6, based on the total amount of quercetin and trans-resveratrol present. In embodiments, the weight to weight ratio of bioperine to trans-resveratrol is from about 0.004 to about 0.04, based on the total amount of bioperine and trans-resveratrol present.

In some embodiments, the composition further comprises Vitamin D, or a derivative thereof.

In embodiments, the composition comprises from about 200 to 300 mg of trans-resveratrol or a derivative thereof. In embodiments, the composition comprises from about 100 to 200 mg of apigenin or a derivative thereof. In embodiments, the composition comprises from about 50 to 150 mg of quercetin or a derivative thereof. In embodiments, the composition comprises from about 1 to 10 mg of bioperine or a derivative thereof. In embodiments, the composition comprises from about 50 to 150 mg of baicalin or a derivative thereof. In embodiments, the composition comprises from about 50 to 150 mg of hesperidin or a derivative thereof. In embodiments, the composition comprises from about 35 to 150 mg of myricetin or a derivative thereof. In embodiments, the composition comprises from about 25 to 75 mg of luteolin or a derivative thereof. In embodiments, the composition comprises from about 10 to 50 mg of Vitamin B3 or a complex thereof. In embodiments, the composition comprises from about 10 to 50 mg of zinc as a salt or complex, based on the metal content present. In embodiments, the composition comprises from about 2000 IU to 7500 IU of Vitamin D or a complex thereof.

In embodiments, the trans-resveratrol, the apigenin, the quercetin, and/or the bioperine are present as one or more glycoside derivatives.

In embodiments, the composition further comprises a carrier, diluent, preservative, or a combination thereof comprising one or more of glucose, fructose, sucrose, maltose, yellow dextrin, white dextrin, aerosol, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.

In embodiments, the composition is in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.

In embodiments, the trans-resveratrol or the derivative thereof, the apigenin or the derivative thereof, the quercetin or the derivative thereof, and/or the bioperine or the derivative thereof are extracts, fractions, compounds, phytochemicals, and/or powders derived from plants.

### Embodiments Listing

Embodiments according to the instant disclosure include:
E1. A composition comprising a biologically active amount of nicotinamide mononucleotide or a derivative thereof, apigenin or a derivative thereof, quercetin or a derivative thereof, and piperine or a derivative thereof.
E2. The composition according to embodiment E1, wherein the weight to weight ratio of apigenin to nicotinamide mononucleotide is from about 0.4 to about 0.8, based on the total amount of apigenin and nicotinamide mononucleotide present.
E3. The composition according to embodiment E1 or E2, wherein the weight to weight ratio of quercetin to nicotinamide mononucleotide is from about 0.2 to about 0.6, based on the total amount of quercetin and nicotinamide mononucleotide present.
E4. The composition according to any one of embodiments E1 through E3, wherein the weight to weight ratio of piperine to nicotinamide mononucleotide is from about 0.02 to about 0.06, based on the total amount of piperine and nicotinamide mononucleotide present.
E5. The composition according to any one of embodiments E1 through E4 further comprising from about 50 to 150 mg of baicalin or a derivative thereof.
E6. The composition according to any one of embodiments E1 through E5 further comprising from about 50 to 150 mg of hesperidin or a derivative thereof.
E7. The composition according to any one of embodiments E1 through E6 further comprising from about 35 to 150 mg of myricetin or a derivative thereof.
E8. The composition according to any one of embodiments E1 through E7 further comprising from about 25 to 75 mg of luteolin or a derivative thereof.
E9. The composition according to any one of embodiments E1 through E8 further comprising from about 10 to 50 mg of Vitamin B3 or a complex thereof.
E10. The composition according to any one of embodiments E1 through E9 further comprising from about 10 to 50 mg of zinc as a salt or complex, based on the metal content present.
E11. The composition according to any one of embodiments E1 through E10 further comprising from about 2000 IU to 7500 IU of Vitamin D or a complex thereof.
E12. The composition according to any one of embodiments E1 through E11 comprising from about 50 mg to 350 mg of nicotinamide mononucleotide or a derivative thereof.
E13. The composition according to any one of embodiments E1 through E12 comprising from about 10 to 200 mg of apigenin or a derivative thereof.
E14. The composition according to any one of embodiments E1 through E13 comprising from about 10 to 200 mg of quercetin or a derivative thereof.
E15. The composition according to any one of embodiments E1 through E14 comprising from about 1 to 20 mg of bioperine or a derivative thereof.
E16. The composition according to any one of embodiments E1 through E15, wherein the nicotinamide mononucleotide, the apigenin, the quercetin, and/or the bioperine are present as one or more glycoside derivatives.
E17. The composition according to any one of embodiments E1 through E16, further comprising a carrier, diluent, preservative, or a combination thereof comprising one or more of glucose, fructose, sucrose, maltose, yellow dextrin, white dextrin, aerosol, cellulose, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.
E18. The composition according to any one of embodiments E1 through E17, in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.
E19. The composition according to any one of embodiments E1 through E18, wherein the nicotinamide mononucleotide or the derivative thereof, the apigenin or the derivative thereof, the quercetin or the derivative thereof, and/or the bioperine or the derivative thereof are extracts, fractions, compounds, phytochemicals, and/or powders derived from plants.
E20. The composition according to any one of embodiments E1 through E19, in the form of an oral dosage suitable for administration to a mammal.
E21. A method to treat a physiological condition of a mammal comprising:
   providing a composition according to any one of embodiments E1 through E20; and administering a biologically effective dosage to the mammal.
E22. The method according to embodiment E21, wherein the physiological condition comprises diabetes, hyperglycemia, hypolipidemia; obesity, hypertension, platelet aggregation; viral infection, atherosclerosis, inflammation, auto-immune disfunction, or a combination thereof.
E23. The method according to embodiment E21, wherein the physiological condition comprises a viral infection.
E24. A composition consisting essentially of a biologically active amount of nicotinamide mononucleotide or a derivative thereof, apigenin or a derivative thereof, quercetin or a derivative thereof, and piperine or a derivative thereof.
E25. The composition according to embodiment E24, including a carrier, a diluent, a preservative, cellulose, dextrin, magnesium stearate, or a combination thereof in the form of a tablet, soft capsule, hard capsule, a pill, or a combination thereof.
E26. The composition according to embodiment E24 or E25 having from about 50 mg to 350 mg of nicotinamide mononucleotide or a derivative thereof, from about 10 mg to 200 mg of apigenin or a derivative thereof, from about 10 mg to 200 mg of quercetin or a derivative thereof, and from about 1 mg to 20 mg of piperine or a derivative thereof.
E27. A composition according to any one of embodiments E1 through E20 comprising a biologically active amount of trans-resveratrol or a derivative thereof, apigenin or a derivative thereof, quercetin or a derivative thereof, and bioperine or a derivative thereof.
E28. A composition comprising a biologically active amount of trans-resveratrol or a derivative thereof, apigenin or a derivative thereof, quercetin or a derivative thereof, and bioperine or a derivative thereof.
E29. The composition according to embodiment E27 or E28, further comprising a biologically active amount of hesperidin, luteolin, EGCG, Vitamin B3, Zinc, or a combination thereof.
E30. The composition according to any one of embodiments E27 through E29, wherein the weight to weight ratio of apigenin to trans-resveratrol is from about 0.4 to about 0.8, based on the total amount of apigenin and trans-resveratrol present, and/or the weight to weight ratio of quercetin to trans-resveratrol is from about 0.2 to about 0.6, based on the total amount of quercetin and trans-resveratrol present.
E31. The composition according to any one of embodiments E27 through E30, wherein the weight to weight ratio of bioperine to trans-resveratrol is from about 0.004 to about 0.04, based on the total amount of bioperine and trans-resveratrol present.
E32. The composition according to any one of embodiments E27 through E31 comprising from about 200 to 300 mg of trans-resveratrol or a derivative thereof.
E33. The composition according to any one of embodiments E27 through E32, comprising from about 100 to 200 mg of apigenin or a derivative thereof.
E34. The composition according to any one of embodiments E27 through E33, comprising from about 50 to 150 mg of quercetin or a derivative thereof.
E35. The composition according to any one of embodiments E27 through E34, comprising from about 1 to 10 mg of bioperine or a derivative thereof.
E36. The composition according to any one of embodiments E27 through E35, further comprising: from about 50 to 150 mg of baicalin or a derivative thereof;
   from about 50 to 150 mg of hesperidin or a derivative thereof;
   from about 35 to 150 mg of myricetin or a derivative thereof;
   from about 25 to 75 mg of luteolin or a derivative thereof;
   from about 10 to 50 mg of Vitamin B3 or a complex thereof;
   from about 10 to 50 mg of zinc as a salt or complex, based on the metal content present;
   from about 2000 IU to 7500 IU of Vitamin D or a complex thereof;
   or a combination thereof.
E37. The composition according to any one of embodiments E27 through E36, wherein the trans-resveratrol, the apigenin, the quercetin, and/or the bioperine are present as one or more glycoside derivatives.
E38. The composition according to any one of embodiments E27 through E37, further comprising a carrier, diluent, preservative, or a combination thereof comprising one or more of glucose, fructose, sucrose, maltose, yellow dextrin, white dextrin, aerosol, cellulose, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.
E39. The composition according to any one of embodiments E27 through E38, in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.
E40. The composition according to any one of embodiments E27 through E39, wherein the trans-resveratrol or the derivative thereof, the apigenin or the derivative thereof, the quercetin or the derivative thereof, and/or the bioperine or the derivative thereof are extracts, fractions, compounds, phytochemicals, and/or powders derived from plants.
E41. A method to treat a physiological condition of a mammal comprising:
   providing a composition according to any one of embodiments E27 through E40; and
   administering a biologically effective dosage to the mammal.
E42. The method according to embodiment E41, wherein the physiological condition comprises diabetes, hyperglycemia, hypolipidemia; obesity, hypertension, platelet aggregation; viral infection, atherosclerosis, inflammation, auto-immune disfunction, or a combination thereof.
E43. The method according to embodiment E41, wherein the physiological condition comprises a viral infection.

The foregoing disclosure and description of the invention is illustrative and explanatory thereof and it can be readily appreciated by those skilled in the art that various changes in the size, shape and materials, as well as in the details of the illustrated construction or combinations of the elements described herein can be made without departing from the spirit of the invention.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

I claim:

1. A composition comprising a biologically active amount of nicotinamide mononucleotide or a derivative thereof, apigenin or a derivative thereof, quercetin or a derivative thereof, and piperine or a derivative thereof.

2. The composition of claim 1, wherein the weight to weight ratio of apigenin to nicotinamide mononucleotide is from about 0.4 to about 0.8, based on the total amount of apigenin and nicotinamide mononucleotide present.

3. The composition of claim 1, wherein the weight to weight ratio of quercetin to nicotinamide mononucleotide is from about 0.2 to about 0.6, based on the total amount of quercetin and nicotinamide mononucleotide present.

4. The composition of claim 1, wherein the weight to weight ratio of piperine to nicotinamide mononucleotide is from about 0.02 to about 0.06, based on the total amount of piperine and nicotinamide mononucleotide present.

5. The composition of claim 1 further comprising:
from about 50 to 150 mg of baicalin or a derivative thereof;
from about 50 to 150 mg of hesperidin or a derivative thereof;
from about 35 to 150 mg of myricetin or a derivative thereof;
from about 25 to 75 mg of luteolin or a derivative thereof;
from about 10 to 50 mg of Vitamin B3 or a complex thereof;
from about 10 to 50 mg of zinc as a salt or complex, based on the metal content present;
from about 2000 IU to 7500 IU of Vitamin D or a complex thereof;
or a combination thereof.

6. The composition of claim 1, comprising from about 50 mg to 350 mg of nicotinamide mononucleotide or a derivative thereof.

7. The composition of claim 1 comprising from about 10 to 200 mg of apigenin or a derivative thereof.

8. The composition of claim 1 comprising from about 10 to 200 mg of quercetin or a derivative thereof.

9. The composition of claim 1 comprising from about 1 to 20 mg of bioperine or a derivative thereof.

10. The composition of claim 1, wherein the nicotinamide mononucleotide, the apigenin, the quercetin, and/or the bioperine are present as one or more glycoside derivatives.

11. The composition of claim 1, further comprising a carrier, diluent, preservative, or a combination thereof comprising one or more of glucose, fructose, sucrose, maltose, yellow dextrin, white dextrin, aerosol, cellulose, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, nicotinamide, calcium pantothenate, amino acids, calcium salts, pigments, flavors, preservatives, distilled water, saline, aqueous glucose solution, ethanol, propylene glycol, polyethylene glycol, vegetable oils, white soft paraffin, paraffin, wax, or a combination thereof.

12. The composition of claim 1, in the form of a tablet, soft capsule, hard capsule, pill, granules, powder, emulsion, suspension, syrup, pellet, food, beverage, an injection solution, drops, suppository, a patch, topical creams, gel food ingredient, inhalable aerosol, or a combination thereof, suitable for administration to a mammal.

13. The composition of claim 1, wherein the nicotinamide mononucleotide or the derivative thereof, the apigenin or the derivative thereof, the quercetin or the derivative thereof, and/or the bioperine or the derivative thereof are extracts, fractions, compounds, phytochemicals, and/or powders derived from plants.

14. The composition of claim 1, in the form of an oral dosage suitable for administration to a mammal.

15. A method to treat a physiological condition of a mammal comprising:
providing a composition of claim 1; and
administering a biologically effective dosage to the mammal.

16. The method of claim 15, wherein the physiological condition comprises:
diabetes, hyperglycemia, hypolipidemia; obesity, hypertension, platelet aggregation; viral infection, atherosclerosis, inflammation, auto-immune disfunction, or a combination thereof.

17. The method of claim 15, wherein the physiological condition comprises a viral infection.

18. A composition consisting essentially of a biologically active amount of nicotinamide mononucleotide or a derivative thereof, apigenin or a derivative thereof, quercetin or a derivative thereof, and piperine or a derivative thereof.

19. The composition of claim 18, including a carrier, a diluent, a preservative, cellulose, dextrin, magnesium stearate, or a combination thereof in the form of a tablet, soft capsule, hard capsule, a pill, or a combination thereof.

20. The composition of claim 19, having from about 50 mg to 350 mg of nicotinamide mononucleotide or a derivative thereof, from about 10 mg to 200 mg of apigenin or a derivative thereof, from about 10 mg to 200 mg of quercetin or a derivative thereof, and from about 1 mg to 20 mg of piperine or a derivative thereof.
